(19) United States
(12) Patent Application Publication
(10) Pub. No.: US 2013/0079416 A1
(43) Pub. Date: Mar. 28, 2013

(54) HOMEOPATHIC MEDICAMENT COMPRISING PHENACETIN FOR THE TREATMENT OF CANCER

(71) Applicants: Christophe Schneider, Aubilly (FR); Caroline Laheurte, St-Loup Sur Semouse (FR); Laurent Martiny, Reims (FR)

(72) Inventors: Christophe Schneider, Aubilly (FR); Caroline Laheurte, St-Loup Sur Semouse (FR); Laurent Martiny, Reims (FR)

(73) Assignee: BOIRON, Sainte Foy les Lyon (FR)

(21) Appl. No.: 13/641,017

(22) PCT Filed: Apr. 7, 2011
(86) PCT No.: PCT/EP2011/055424

§ 371 Date: Dec. 14, 2012

(30) Foreign Application Priority Data
Apr. 14, 2010 (FR) 1052831

(51) Int. Cl. A61K 31/167; A61K 45/06

(52) U.S. Cl. CPC A61K 31/167; A61K 45/06
USPC 514/629; 564/223

(57) ABSTRACT

The present invention relates to a composition comprising phenacetin, in particular a Phenacetinum 4CH homeopathic medicament for use in the treatment of cancer.

### FIELD OF THE INVENTION

The present invention relates to the field of the medicaments used in the present invention and/or the treatment of cancers, including in particular melanomas, carcinomas, thyroid cancers, colorectal cancers and cancers of the breast, prostate and lung.

In particular, the invention relates to an anti-cancer homeopathic medicament comprising phenacetin.

The anti-cancer medicaments in question are those with a mode of action based in particular on inhibiting tumour invasion and tumour growth.

Other subjects of the present invention are the use of phenacetin in the treatment of cancer, and more particularly in the treatment of the different forms (or stages) of melanoma, and the use of phenacetin in the manufacture of a medicament for treating cancer.

Different pharmaceutical forms, different administration routes and different drug associations can be envisaged within the framework of the invention.

### TECHNOLOGICAL BACKGROUND

Melanoma ranks second in human cancers in terms of the number of years of life lost. Over the last ten years, its incidence has increased more (3 to 5% per year) than the other types of cancer, with the exception of bronchial cancer in women. In 2000, it was estimated that one individual in 100 would develop a melanoma in the course of their life and that one patient in 2 would be younger than 50 years old. This type of cancer is inescapably becoming a major public health problem. Melanoma is a malignant tumour having a very bad prognosis with a high risk of lymph node and visceral metastases.

Over the last few years, the tumour mechanism has been studied and the mechanisms underlying the tumour progression have been explained little by little. Thus, during the tumour progression, the cancer cells leave the primary tumour, pass through the vascular membranes and migrate into the surrounding extracellular matrix.

These phenomena together involve the secretion, then the activation, of proteolytic enzymes, such as Matrix MetalloProteinases (MMPs) or the plasminogen activation system (Hornebeck W. et al.). The expression profile of these proteolytic enzymes, studied in various cell lines of human or mouse melanoma, reveals a very strong increase in the expression of various MMPs, in a manner correlated with the invasive phenotype of these cells (Egeblad M. et al.). The expression on the cell surface of MMP-2, functionally active, directly influences the adhesion and the spread of the cells on the components of the extracellular matrix and of the basement membranes, and promotes the migration and invasion of these cells.

The activation of MMP-2 requires complexation with its inhibitor, TIMP-2, and another transmembrane MMP, MT1-MMP or MMP-14 (Egeblad M. et al.). It also requires the presence of integrin [alpha]V[beta]3 the expression of which increases with the degree of invasivity of the melanoma cells. This integrin acts as a membrane receptor of MMP-2, promotes its activation and focuses the active form of MMP-2 on the lamellipodia, which induce progression of the cancer cell inside the extracellular matrix (Seftor REB et al.; Deryugina E1 et al.). Moreover, in the course of the tumour progression, several inflammatory phenomena occur which involve the activation of inflammatory cells (neutrophil granulocytes, monocytes, macrophages, etc.) leading to the release of various cytokines and growth factors, but also of MMPs, such as MMP-9.

Several active ingredients are already known which are effective to a greater or lesser extent in the treatment of different forms of cancer.

The present invention forms part of the research into new treatments for cancer in its different forms, using active pharmaceutical ingredients known for other indications.

In this context, the inventors became interested in phenacetin.

Discovered in 1887, phenacetin is an aromatic organic compound also found under other names, in particular acetophenetidin or N-(4-ethoxyphenol)acetamide. It is present in the form of a fine powder composed of bright white crystals.

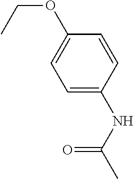

It was indicated for pain relief Phenacetin has a central and/or peripheral analgesic action and an antipyretic action. These two activities are comparable in intensity and duration to those of paracetamol and are due to the phenacetin itself as well as to its metabolite, paracetamol.

The analgesic effect is due to the action of phenacetin on the sensory areas of the spinal cord. Moreover, phenacetin has a sedative action on the heart, where it acts as a negative isotrope. This is also an antipyretic which acts on the brain in order to lower the temperature. It is also used to treat rheumatoid arthritis, intercostal neuralgia and certain forms of ataxia.

Phenacetin administered orally over long periods (96 weeks) and at high doses (food composed of approximately 1% phenacetin) has toxic effects and causes benign and malignant tumours of the urinary tract in mice (Nakanishi et al., 1982) and rats (Muradian, 1986), and of the nasal cavity in rats (IARC Monographs, 1980). In rats, phenacetin also administered over a long time and at very high doses, alone or in combination with phenazone, slightly increases the incidence of renal tumours (Johansson, 1981). The rats treated with a mixture of phenacetin, phenazone and caffeine develop hepatomas (Johansson, 1981). Similarly, in rats, phenacetin increases the incidence of chemically induced bladder tumours (IARC Monographs, 1980).

TABLE 1

| | | | Route | Lowest | |
|---|---|---|---|---|---|
| | | | of expo- | toxic dose | |
| Toxicity | Substance | Species | sure | (TDL₀)* | Comments |
| Multiple | phenacetin | Rodent (rat) | Oral | 10 mg/kg/ | Blood- |
| doses | | | | 4 weeks | methemoglobin- |
| | | | | (inter- | emia-carboxy- |
| | | | | mittent) | haemoglobin |
| | | | | | Blood-changes: |
| | | | | | count of |
| | | | | | other blood cells |
| | | | | | (not specified) |
| | | | | | Blood-changes: |
| | | | | | erythrocyte count |

*TDL₀: the lowest published toxic dose

With regard to acute toxicity in humans, the symptoms marking an overdose (dose greater than 3 g by the oral route) (Winek et al., 2001) of phenacetin are as follows:
cyanosis secondary to methemoglobin formation;
functional anaemia able to trigger a cardiovascular collapse and angina crises;
respiratory depression;
cutaneo-mucous eruptions and hyperthermia;
central disorder manifesting itself by prostration or a hallucinatory state preceding a coma;
possible hepatic cell disorder.

To date, no data are available on the toxicity of phenacetin when it is administered by the nasal route or by injection.

With regard to chronic toxicity, a daily consumption of phenacetin over several years can lead to renal disorders such as papillary necroses and secondary tubulo-interstitial nephritis followed by renal failure. The first symptoms of this nephropathy can appear after daily administration of 1 gram per day (by the oral route) for 3 years. Epidemiological studies have shown a genetic predisposition linked to the presence of the HLA-B12 antigen. The renal disorder is often complicated by arterial hypertension and a state of premature and severe atherosclerosis. The toxic cell injury is attributed to the toxicity of the transformation products of phenacetin and of phenetidine.

There are few data on the carcinogenicity of phenacetin in humans, as this substance was most often consumed mixed with other substances. According to the International Agency for Research on Cancer (IARC), there are several reports of cases of renal tumours in patients who have consumed large quantities of analgesics also containing phenacetin (IARC, 1980; Porpaczy and Schramek, 1981). A study of 569 patients suffering from renal cancer shows that the long-term administration (>3 years) of products containing, among other things, phenacetin is associated with a twofold increased risk for renal cancer (McLaughlin et al., 1985). Similarly, another study shows that the consumption of analgesics containing phenacetin increases the risk of developing cancer of the bladder in women (Piper et al., 1985). Phenacetin is currently listed as a carcinogen by the IARC.

TABLE 2

| | | | Route | Lowest | |
|---|---|---|---|---|---|
| | Sub- | | of expo- | toxic dose | |
| Toxicity | stance | Species | sure | (TDL₀)* | Comments |
| acute | Phen- | Human | Not indicated | 74 mg/kg | The details |
| | acetin | | | | of the toxic |
| | | | | | effects other than |
| | | | | | the lethal dose |
| | | | | | have not yet |
| | | | | | been indicated |
| Multiple | Phen- | Human | Oral | 80 mg/kg/63 | Tumorigenic- |
| doses | acetin | | | years | carcinogenic |
| | | | | (intermittent) | according |
| | | | | | to RTECS |
| | | | | | criteria Kidney/ |
| | | | | | ureter/bladder- |
| | | | | | Renal tumours |

Moreover, it is to be noted that phenacetin was withdrawn from the market in 1983, as a result of its link with cases of nephropathies.

Despite all of these pharmacological preconceptions unfavourable to phenacetin, the inventors became interested in this molecule in the treatment of cancer.

### BRIEF DESCRIPTION OF THE INVENTION

In a very surprising and unexpected manner, the inventors have thus been able to demonstrate that phenacetin could be used, in particular in a homeopathic dilution, to treat cancer in animals and more especially in humans.

Overcoming the preconceptions, the inventors have discovered that phenacetin in a very dilute form, for example in the form of a Phenacetinum 4CH homeopathic medicament, more particularly inhibits tumour invasion and tumour growth or development.

In this way, the present invention relates to a pharmaceutical composition comprising phenacetin for use as an anti-cancer medicament.

The composition according to the invention is therefore a composition comprising phenacetin for use in the prevention and/or the treatment of cancer.

### Definitions

These definitions are given by way of non-limiting examples.

Throughout the present disclosure, “any” singular denotes either a singular or a plural. Within the meaning of the invention, the term “phenacetin” denotes not only the aromatic organic compound N-(4-ethoxyphenyl)acetamide, CAS number 62-44-2, but also its analogues such as paracetamol or N-(4-hydroxyphenyl)ethanamide, CA number 102-90-2, and N-(4-alkoxyphenyl)acetamides.

Within the meaning of the present invention, a homeopathic medicament is defined according to directive 2001/83/EC of the European Parliament and of the Council. A homeopathic medicament is obtained from products, substances or compositions called homeopathic stocks of plant or animal origin or chemical products (mineral or organic), by the successive dilution method. This dilution is carried out by successive operations of dividing the stock in a vehicle (dilution or trituration) at 1/100 (centesimal) or at 1/10 (decimal), and by successive dynamization operations. The number of operations carried out in this way defines the degree of dilution.

Thus, homeopathic medicaments are usually designated by the Latin name of the stock followed by an indication of the degree of dilution.

### DETAILED DESCRIPTION OF THE INVENTION

Advantageously, the composition according to the invention is indicated for the prevention and/or the treatment of a cancer, preferably chosen from the group constituted by melanomas, carcinomas, thyroid cancers, colorectal cancers and cancers of the breast, prostate and lung.

Another subject of the present invention is a composition comprising phenacetin for use as an agent inhibiting tumour development and/or the tumour invasion process.

In fact, the composition according to the invention is effective in the inhibition of the proliferation of tumour cells (and more particularly of cells from melanomas). In the presence of the composition, the percentage proliferation of the cells observed in vitro and preferably in vivo is reduced by at least 20, 30, 40 or 50% with homeopathic concentrations.

The inhibition of the proliferation and migration of tumour cells can be tested as indicated below in the examples.

The composition according to the invention is capable of reaching the target cells after its administration, in order to exert its biological action.

Moreover, the composition according to the invention is able to inhibit the expression of certain genes coding for proteins involved in tumour invasion, in particular the MMP-2 and MMP-14 genes. It therefore follows that another subject of the invention is a composition comprising phenacetin for use as an agent inhibiting the expression of the MMP-2 gene and/or MMP-14 gene coding for the Matrix MetalloProteinases MMP-2 & MMP-14 respectively.

Preferably, the composition according to the invention comprises an aqueous, alcoholic or hydroalcoholic solution of phenacetin. Still more preferably, the composition according to the invention is constituted by such a solution.

According to another advantageous embodiment of the invention, the composition is obtained from an aqueous, alcoholic or hydroalcoholic solution of phenacetin.

For example, this solution can be used to impregnate a solid form of excipient such as a neutral sugar granule, among other things.

According to a remarkable feature of the invention, the composition is a dilute solution with a phenacetin concentration Cp less than or equal to 200 μg/ml, preferably 1 μg/ml, and, still more preferably comprised between 1 and 50 ng/ml, ideally between 5 and 15 ng/ml.

Still more preferably, this solution is a homeopathic medicament, preferably Phenacetinum 2CH, 3CH, 4CH or 5CH, preferably Phenacetinum 4CH.

In order to improve its pharmaceutical activity, the composition according to the invention can moreover comprise at least one other molecule that is pharmaceutically active in the prevention and/or the treatment of cancer.

This improvement in pharmaceutical activity can be reflected in an increased inhibitor effect vis-à-vis tumour development and/or the tumour invasion process, or vis-à-vis the expression of the MMP-2 and MMP-14 genes or vis-à-vis the remission of the disease in cancer patients or vis-à-vis the limitation of side effects, or vis-à-vis the prolonging of the activity over time for a given administered dose, among other things.

The other molecule that is pharmaceutically active in the prevention and/or the treatment of cancer can be any molecule capable of participating in the treatment of cancer, such as for example a chemotherapeutic agent and/or immunotherapeutic agent. There can be mentioned as examples: dacarbazine, cisplatin, vindesine, fotemustine, nitrosoureas or cyclic peptides, in particular those according to WO 2007/107654, a tumour epitope known to be specifically associated with tumour cells and particularly a tumour epitope associated with melanoma cells such as the MAGE 1, 2 and 3, BAGE, GAGE 1 and 2
antigens or melanocytic differentiation antigens such as tyrosinase and Melan-A/MART-1, or also interleukin 2 or interferon α.

In the same way, the present invention also relates to any drug association comprising the composition as defined in the present application and at least one homeopathic or allopathic medicament.

The latter can comprise an active ingredient of the type of those mentioned above.

Within the context of combining the association of the composition according to the invention with other pharmaceutically active molecules (homeopathic or allopathic medicaments), it can be envisaged to administer them together or separately in time.

It could also be advisable for the associated medicament to be a homeopathic medicament, for example Ruta graveloens (6 CH, 9CH or 200 CH), Sabal serrulata (e.g. 200 CH) or Lycopodium or Calcarea phosphorica (e.g. 3 CH), alone or combined with one another. As regards galenics, this composition according to the invention can be formulated for administration by the oral, nasal, intramuscular, intravenous or subcutaneous route. Advantageously, this composition can moreover comprise a pharmaceutically acceptable excipient or a carrier.

Another subject of the invention is the use of the composition according to the invention to obtain (manufacture) an anti-cancer medicament or a medicament intended for a preventive and/or therapeutic use based on inhibiting tumour development and/or the tumour invasion process and/or the expression of the MMP-2 gene and/or MMP-14 gene coding for the Matrix MetalloProteinases MMP-2 & MMP-14 respectively.

For such a use, the medicament is administered by the oral, nasal, intramuscular, intravenous or subcutaneous route, preferably by the oral route.

Another subject of the present invention relates to a method comprising the in vivo administration of the composition as defined in the present application, for the prevention and/or the treatment of various types of cancer, for example a cancer chosen from the group constituted by melanomas, carcinomas, thyroid cancers, thyroid cancers, colorectal cancers and cancers of the breast, prostate and lung.

The invention therefore relates to a method for treating cancer consisting of administering the composition as defined in the present application.

By “treating cancer” is meant both direct treatment of tumours, for example by reducing or stabilizing their number or their size (curative effect), while also preventing the in situ progression of the tumour cells or their diffusion, or the establishment of tumours, and treatment of the harmful effects linked to the presence of these tumours, in particular attenuation of the symptoms observed in a patient or improvement in their quality of life. The invention also relates to the in vitro or in vivo use of the composition as defined in the present application to inhibit tumour development and/or the tumour invasion process at different stages.

Thus, the composition according to the invention can be used to treat primary tumours, optionally before surgical exeresis, or to treat the surrounding areas in order to limit the first stage of invasion by local cutaneous metastases.

The composition according to the invention is very suitable for administration by the oral route, for example:
in solid form: granules of excipient(s)—e.g. sugar—or compressed tablets impregnated with a solution of phenacetin, for example Phenacetinum 4CH;
or in liquid form: ingestible or injectable solution.

### DESCRIPTION OF THE FIGURES

FIG. 1 shows a histogram giving the fluorescence intensity of the invasive cells as a function of the treatment with Phenacetinum 4CH or the treatment with the control (cf. Example 3). The fluorescence is measured with a spectrofluorometer and is in arbitrary units (AU). The fluorescence is proportional to the tumour invasion.

FIG. 2 shows a histogram giving the relative genomic expression of the Matrix MetalloProteinases MMP-2 and MMP-14 expressed in tumour cells treated with Phenacetinum 4CH compared with tumour cells treated with the control (cf. Example 4).

FIG. 3 shows a graph giving the tumour volume (mm³) for cancerous mice treated with the solution of Phenacetinum 4CH and with the control (cf. Example 5).
NB: p<0.05 indicated in FIGS. 1 to 3 corresponds to the statistical threshold of significance p <0.05.

FIG. 4 shows a graph giving the survival percentage as a function of time in days for cancerous mice treated with Phenacetinum 4CH and with the control (cf. Example 5).

### EXAMPLES

The examples which follow describe the preparation of a Phenacetinum 4CH homeopathic medicament, and a control, the measurement of the effect of Phenacetinum 4CH on in vitro tumour invasion and on the genomic expression of MMP-2 and MMP-14, and the measurement of the effect of Phenacetinum 4CH on in vivo tumour growth in mice presenting a form of murine melanoma.

### 1. Method of Preparation of Phenacetinum 4CH

Phenacetinum 4CH is a homeopathic medicament prepared in accordance with monograph 2371 of European Pharmacopoeia 6.1 of 2008 entitled Methods of preparation of homeopathic stocks and potentisation. Phenacetinum 4CH is prepared from Phenacetin (starting material) which meets the requirements of the Homeopathic Preparations monograph (1038). In fact, 5.09 g phenacetin is dissolved in 438.4 g alcohol 70% v/v (Phenacetinum 1CH) accompanied by magnetic stirring. Then, 0.3 ml Phenacetinum 1CH is added to 29.70 ml sterile water (Fresenius) in a 45.0-ml square bottle under a laminar flow hood. In order to obtain Phenacetinum 2CH, this solution is then stirred according to the recommendations of the Pharmacopée Française, Xth edition, 6th supplement of 1983. Then, 0.3 ml Phenacetinum 2CH is added to 29.70 ml sterile water in a 45.0-ml square bottle under a laminar flow hood, then the solution is stirred in the same manner as during the preceding stage in order to obtain Phenacetinum 3CH. Finally, 0.84 ml Phenacetinum 3CH is added to 83.16 ml sterile water in a 125-ml round bottle under a laminar flow hood and this solution is stirred in the same manner as during the preceding stage in order to obtain Phenacetinum 4CH. This solution is then filtered with 0.2-μm filters.

2-ml ampoule bottles are sterilized in an oven (160° C. for 2 hours) then filled with Phenacetinum 4CH under a laminar flow hood and under vacuum.

### 2. Method of Preparation of the Control

The control used in the experiments corresponds to sterile water (Fresenius). 2-ml ampoule bottles are sterilized in an oven (160° C. for 2 hours) then filled with this sterile water under a laminar flow hood.

### 3. Measurement of the Effect of Phenacetinum 4CH on In Vitro Tumour Invasion

The effects of Phenacetinum 4CH on human thyroid carcinoma cell invasion were measured in Matrigel-coated Boyden chambers following the experimental protocol described below. Invasive human thyroid carcinoma cells, FTC-238 (Human follicular thyroid carcinoma), are seeded randomly at a rate of 50,000 cells/culture well in a volume of 500 μl in 24-well plates.

After adhesion of the cells (24h), they are treated with Phenacetinum 4CH or with the control according to the following protocol: 100 μl/well at TO (D1), 50 μl/well at T6h (D1) and 100 μl/well at T24h (D2).

At T30h (D2), the cells are recovered by trypsinization (100 μl/well), centrifuged (1200 rpm, 5 min) and resuspended in 100 μl medium without serum. The cells are then placed in cell invasion plates (CellBiolabs 96-well plates ref. CBA-112). The membrane is then recovered and rehydrated by addition of 100 μl medium without serum at 37° C. over 1 h at ambient temperature. The cell invasion is measured using medium containing 10% serum as chemoattractant. The resuspended cells are then added to the culture wells containing either 20 μl Phenacetinum 4CH or 20 μl of the control solution. The culture plate is incubated in an oven for 16 h.

The cells that have passed through the membrane are dissociated and lysed. They are then stained with a dye (CyQuant G R dye, CellBiolabs) and the culture plate is read with a spectrofluorometer (Perkin Elmer LSB) at 480/520 nm.

Each test is carried out in triplicate for each handling series.

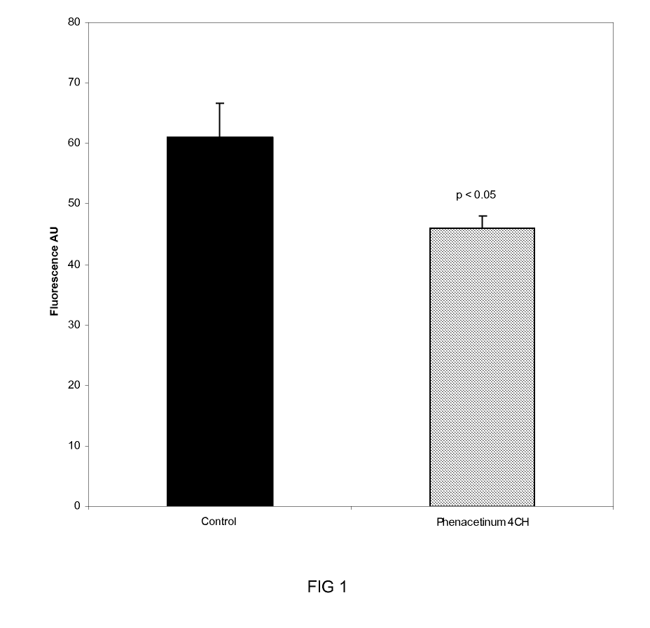

The main result is presented in the attached FIG. 1.

The results show a statistically significant inhibitor effect of Phenacetinum 4CH on the tumour invasion process compared with the cells treated with the control solution.

### 4. Measurement of the Effect of Phenacetinum 4CH on Genomic Expression

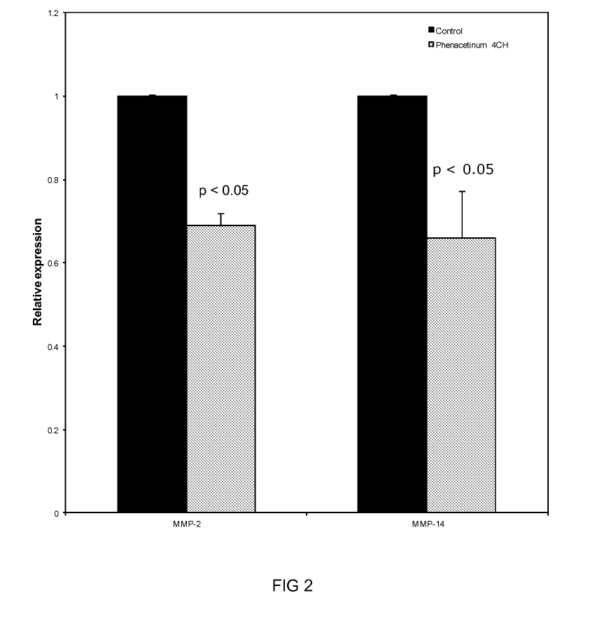

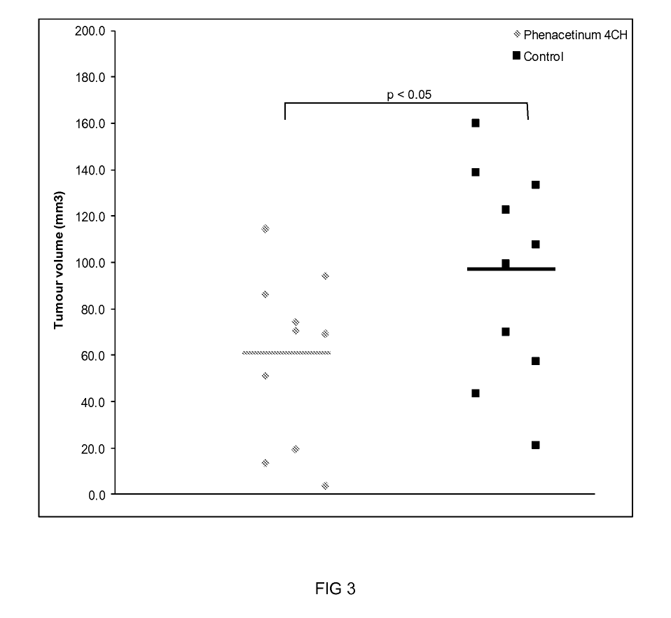

The genomic expression of 2 agents involved in tumour invasion (MMP-2 and MMP-14) were studied by quantitative PCR (qPCR). The results presented in the attached FIG. 2 reflect 3 independent experiments each carried out in triplicate.

It appears that Phenacetinum 4CH inhibits the expression of 2 metalloproteases (MMP-2 and MMP-14) of the FTC-238 cells compared with these same cells treated with the control solution.

### 5. Measurement of the In Vivo Effect of Phenacetinum 4CH

The effects of Phenacetinum 4CH on the in vivo invasion process are studied in the following manner. The mice used belong to the C57BL6 line and are grouped in batches of 10 individuals. On D0, they are injected subcutaneously in the left flank with a 100-μl volume of a solution containing 2.5 10⁵ B16F1 cells (murine melanoma cells). The injection solution contains NaCl0.85%.

In the control group, each mouse is injected each morning with 100 μl sterile water contained in a single-use ampoule by the intraperitoneal (IP) route at a rate of one injection/day throughout the monitoring of the tumour progression.

In the treated group, each mouse is injected each morning with 100 μl Phenacetinum 4CH contained in a single-use ampoule by the intraperitoneal (IP) route at a rate of one injection/day throughout the monitoring of the tumour progression.

The effect of Phenacetinum 4CH in the treated group is evaluated by measurement of the kinetics of the tumour volume and compared with the control group. The volume of the tumour is determined according to the following formula: v=½ A×B² where A corresponds to the largest dimension of the tumour, while B corresponds to the smallest dimension of the tumour (Wald et al., 2001).

The effect of Phenacetinum 4CH in the treated group is also evaluated by calculation of the survival time of the mice compared with the control group, knowing that the size of the tumour cannot exceed 1000 mm³. A mouse in which the volume exceeds 1000 mm³ is sacrificed and considered dead.

For this in vivo study, the tumour volumes of the group treated with Phenacetinum 4CH and of the control group are compared using a non-parametric Mann and Whitney u-test. The result of the effect of Phenacetinum 4CH on in vivo tumour progression is presented at D14 (i.e. 14 days after injection of the tumour cells) in the attached FIG. 3.

Phenacetinum 4CH slows the tumour development in a statistically significant manner compared with a control treatment.

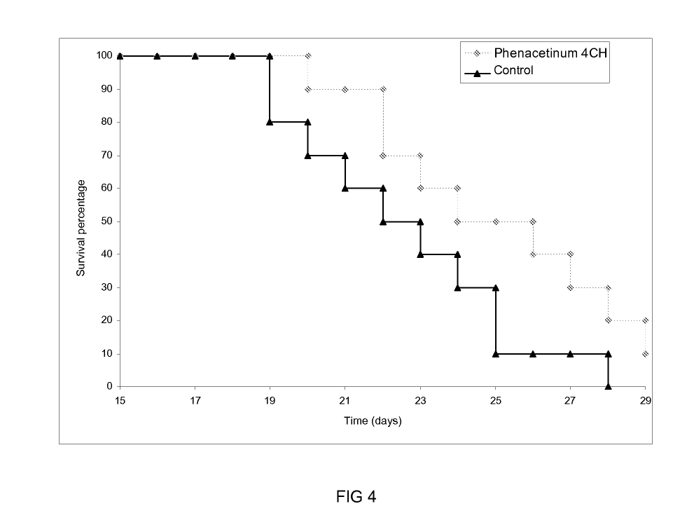

The result of the effect of Phenacetinum 4CH on the survival of the animals is presented in the attached FIG. 4. It is shown that Phenacetinum 4CH improves the survival time of the animals compared with the control group treated with H2O.

It appears that Phenacetinum 4CH inhibits tumour progression during tests carried out in vitro and in vivo.

The composition according to the invention is very suitable for administration by the oral route, for example granules of excipient or compressed tablets impregnated with a solution of phenacetin, for example Phenacetinum 4CH or ingested or injected solution in a liquid.

### BIBLIOGRAPHIC REFERENCES

W. Hornebeck, H. Emonard, J. C. Monboisse and G. Bellon. Matrix-directed regulation of pericellular proteolysis and tumor progression. Sem. Cancer Biol. 12 (2002), pp. 231-241.
M. Egeblad, Z. Werb. New functions for the matrix metalloproteinases in cancer progression. Nat Rev Cancer (2002), pp. 161-74
R. E. B. Seftor, E. A. Seftor, K. R. Gehlen, W. G. Stetler-Stevenson, P. D. Brown, E. Ruoslathi and M. J. C. Hendrix. Role of the αVβ3 integrin in human melanoma cell adhesion. Proc. Natl. Acad. Sci. USA 89 (1992), pp. 1557-1561.
E. I. Deryugina, B. Ratnikov, E. Monosov, T. L. Postnova, R. DiScipio, J. W. Smith and A. Y. Strongin. MT1-MMP initiates activation of pro-MMP-2 and integrin αVβ3 promotes maturation of MMP-2 in breast carcinoma cells. Exp. Cell Res. 263 (2001), pp. 209-223.
Nakanishi K, Kurata Y, Oshima M, Fukushima S, Ito N. Carcinogenicity of phenacetin: long-term feeding study in B6c3f1 mice. Int J Cancer. 1982 Apr 15;29(4):439-44.
Muradian RE. Experimental study of the carcinogenicity of phenacetin. Vopr Onkol. 1986;32(5):63-70.
IARC Monographs supplement 7, Phenacetin (Group 2A) and analgesic mixtures containing phenacetin (Group 1) 1980
Johansson S L. Carcinogenicity of analgesics: long-term treatment of Sprague-Dawley rats with phenacetin, phenazone, caffeine and paracetamol (acetamidophen). Int J Cancer. 1981;27(4):521-9.
Yamamoto R S, Frankel H H, Weisburger E K. Effect of phenacetin and N-alkylacetanilides on N-2-fluorenylacetamide hepatocarcinogenesis. Cancer Lett. 1979 Dec;8(2): 183-8.
Winek C. L., Wahba W. W., Winek C. L. Jr., Winek Balzer T. Drugs and chemical blood-level data 2001. Forensic Science International 122 (2001) 107-123.
IARC Monographs supplement 7, Phenacetin (Group 2A) and analgesic mixtures containing phenacetin (Group 1) 1980
Porpáczy P, Schramek P. Analgesic nephropathy and phenacetin-induced transitional cell carcinoma—analysis of 300 patients with long-term consumption of phenacetin-containing drugs. Eur Urol. 1981;7(6):349-54.
McLaughlin J K, Blot W J, Mehl E S, Fraumeni J F Jr. Relation of analgesic use to renal cancer: population-based findings. Natl Cancer Inst Monogr. 1985 Dec; 69: 217-22.
Piper J M, Tonascia J, Matanoski G M. Heavy phenacetin use and bladder cancer in women aged 20 to 49 years. N Engl J Med. 1985 Aug 1; 313(5):292-5.
RTCES National Institute for occupational safety and health. Centre canadien d'hygiène et de sécurité au travail, Version 2009-1.
Wald M, Olejar T, Sebkova V, Zadinova M, Boubelik M, Pouckova P. Mixture of trypsin, chymotrypsin and papain reduces formation of metastases and extends survival time of C57B16 mice with syngeneic melanoma B16. Cancer Chemother Pharmacol. 2001 Jul 47; 47:S16-22.

1. A composition comprising phenacetin capable of being used as an anti-cancer medicament.

2. The composition according to claim 1, for prevention and/or treatment of a cancer optionally selected from the group consisting of melanomas, carcinomas, thyroid cancers, colorectal cancers and cancers of the breast, prostate and lung.

3. The composition according to claim 1, capable of being used as an agent inhibiting tumour growth and/or a tumour invasion process.

4. The composition according to claim 1, capable of being used as an agent inhibiting expression of the MMP-2 gene and/or MMP-14 gene coding for the Matrix MetalloProteinases MMP-2 & MMP-14 respectively.

5. The composition according to claim 1, wherein said composition comprises an aqueous, alcoholic or hydroalcoholic solution of phenacetin.

6. The composition according to claim 5, wherein said composition comprises an aqueous, alcoholic or hydroalcoholic solution of phenacetin.

7. The composition according to claim 1, wherein said composition is obtained from an aqueous, alcoholic or hydroalcoholic solution of phenacetin.

8. The composition according to claim 5, wherein said solution is a dilute solution with a phenacetin concentration Cp less than or equal to 200 μg/ml, optionally 1 μg/ml.

9. The composition according to claim 5, wherein said solution is a homeopathic medicament, optionally Phenacetinum 2CH, 3CH, 4CH or 5CH.

10. The composition according to claim 1, wherein said composition comprises at least one other molecule that is pharmaceutically active in prevention and/or treatment of cancer.

11. The composition according to claim 1, wherein said composition is formulated for administration by an oral, nasal, intramuscular, intravenous or subcutaneous route.

12. The composition according to claim 1, capable of being used to obtain an anti-cancer medicament or a medicament intended for a preventive and/or therapeutic use based on inhibiting tumour development and/or tumour invasion process and/or expression of the MMP-2 gene and/or MMP-14 gene coding for Matrix MetalloProteinases MMP-2 & MMP-14 respectively.

13. A medicament comprising a composition according to claim 12, wherein said medicament is suitable to be administered by an oral, nasal, intramuscular, intravenous and/or subcutaneous route, optionally by an oral route.

14. Drug association comprising said composition according to claim 1 and at least one homeopathic or allopathic medicament.

15. A method for prevention and/or treatment of a cancer optionally selected from the group consisting of melanomas, carcinomas, thyroid cancers, colorectal cancers and cancers of the breast, prostate and lung, said method comprising administering a composition of claim 1 to a subject in need thereof.

16. A method for inhibiting tumour growth and/or a tumour invasion process, said method comprising administering a composition of claim 1 to a subject in need thereof.

17. A method for inhibiting expression of the MMP-2 gene and/or MMP-14 gene coding for the Matrix MetalloProteinases MMP-2 & MMP-14 respectively, said method comprising administering a composition of claim 1 to a subject in need thereof.
